(19) United States
(12) Patent Application Publication
(10) Pub. No.: US 2023/0002755 A1
(43) Pub. Date: Jan. 5, 2023

(54) METHOD FOR PRODUCING NON-RIBOSOMAL RNA-CONTAINING SAMPLE

(71) Applicant: RIKEN, Saitama (JP)

(72) Inventors: Shintaro IWASAKI, Saitama (JP); Mari MITO, Saitama (JP)

(73) Assignee: RIKEN, Saitama (JP)

(21) Appl. No.: 17/778,749

(22) PCT Filed: Nov. 24, 2020
(86) PCT No.: PCT/JP2020/043538

§ 371 Date: May 20, 2022

(30) Foreign Application Priority Data
Nov. 25, 2019 (JP) 2019-211933

(51) Int. Cl. C12N 15/10; C12Q 1/6869

(52) U.S. Cl. CPC C12N 15/1017; C12Q 1/6869; C12N 15/1062

(57) ABSTRACT

An object of the present invention is to provide a method for producing a non-ribosomal RNA-containing sample, which comprises a novel step for removing ribosomes. According to the present invention, there is provided a method for producing a non-ribosomal RNA-containing sample, which comprises the step (a) of splitting subunits of ribosomes and mRNAs in a sample containing mRNAs and ribosomes, and the step (b) of removing the subunits of ribosomes split in the step (a).

### TECHNICAL FIELD

The present invention relates to a method for producing a non-ribosomal RNA-containing sample (sample containing a non-ribosomal RNA). The present invention relates to a method for analyzing a non-ribosomal RNA using a non-ribosomal RNA-containing sample produced by the method for producing a non-ribosomal RNA-containing sample. The present invention further relates to a kit used for carrying out the method for producing a non-ribosomal RNA-containing sample.

### CROSS-REFERENCE TO RELATED APPLICATION

This application claims the conventional priority based on Japanese Patent Application No. 2019-211933, filed on Nov. 25, 2019, of which entire disclosures are incorporated herein by reference.

### BACKGROUND ART

The advent of next-generation sequencers has dramatically increased the speed of sequence analysis, allowing studies targeting large genomic regions. Next-generation sequencers can simultaneously process several tens to hundreds of millions of randomly truncated DNA fragments in parallel, yielding data ranging from one billion bases (1 gigabases) to 100 billion bases (1 terabases) in a single sequencing run. In addition to whole genome sequencing, next-generation sequencers are used for targeted sequencing, which targets genomic regions relating to specific research targets such as diseases, epigenetic researches such as methylation sequencing, and so forth.

Next-generation sequencers are also used to study the central dogma of molecular biology, namely, the concept that genetic information is transmitted in the order of “DNA→(transcription)→mRNA (messenger RNA)→(translation) protein”. For example. RNA-Seq (RNA sequencing) using a next-generation sequencer can reveal the presence and amount of RNAs in a biological sample at a specific moment, and thereby enables comprehensive gene expression analysis. Furthermore, in researches to elucidate how the reaction called translation. in which proteins are produced from mRNA, is regulated. a technique called ribosome profiling using a next-generation sequencer enables to comprehensively analyze which codons of what kind of mRNA are decoded by ribosomes to give a bird's-eye view over the state of translation.

The ribosome profiling is a technique based on deep sequencing of mRNA fragments protected by ribosomes (Patent document 1). Information from ribosome profiling can be used for investigation of translation regulation, measurement of gene expression, measurement of protein synthesis rates, or prediction of abundance of proteins.
Patent document 1: U.S. Pat. No. 8,486,865
Non-patent document 1: Ingolia et al., 2009. Science, 324, 218-23
Non-patent document 2: Weinberg et al., 2016, Cell Rep., 14, 1787-1799
Non-patent document 3: McGinley and Ingolia, 2017, Methods, 126, 112-129

The entire disclosures of Patent document 1 and Non-patent documents 1 to 3 are incorporated herein by reference.

### SUMMARY OF THE INVENTION

### Object to be Achieved by the Invention

Conventional ribosome profiling has the problem that ribosomal RNA (rRNA) excessively occupies the sequencing library, resulting in a low percentage of sequence reads available for analysis, specifically, low percentage of sequence reads mapped on protein coding regions (CDS). For example. Non-patent document 1 reported that, as a result of sequencing of 42 million fragments obtained by using the ribosome protection assay for budding yeast Saccharomyces cerevisiae, it was found that 7 million (16%) sequence reads were mapped on CDS, but most of the rest were derived from rRNA. In general, statistical analysis depends on the scale of mRNA sequence reads, and therefore low yields of sequence reads available in libraries can hinder analysis in deep sequencing-based approaches such as ribosome profiling.

In addition to the ribosome profiling, RNA-seq also suffers from the problem of contamination of excessive rRNAs in sequencing libraries. Non-patent document 2 reported that when a library for RNA-seq was prepared from total RNA in which mRNAs were not concentrated, 90.2% of the 199.7 million reads were derived from rRNAs. The presence of excessive rRNA sequencing reads imposes a problem that it significantly reduces the efficiency of transcriptome analysis.

To reduce rRNA sequence reads, rRNA-subtraction oligonucleotides, which can hybridize to rRNAs to trap them on magnetic beads, have been used (Non-patent documents 2 and 3). However, even rRNA-depletion using rRNA-subtraction oligonucleotides could not sufficiently reduce rRNA sequence reads. Therefore, a novel method for reducing rRNA sequence reads has been desired.

Therefore, an object of the present invention is to provide a method for producing a non-ribosomal RNA-containing sample, which method comprises a novel step for removing ribosomes.

### Means for Achieving the Object

The inventors of the present invention conducted various researches in order to achieve the above object, and as a result, found that ribosomal subunits can be efficiently removed by splitting ribosomal subunits and mRNAs. The present invention was accomplished on the basis of this finding.

The present invention provides the following inventions.
[1] A method for producing a non-ribosomal RNA-containing sample, which comprises the step (a) of splitting subunits of ribosomes and mRNAs in a sample containing mRNAs and ribosomes, and the step (b) of removing the subunits of ribosomes split in the step (a).
[2] The method for producing a non-ribosomal RNA-containing sample according to [1], which further comprises the step of degrading RNAs or fragmenting RNAs in a sample containing mRNAs and ribosomes.
[3] The method for producing a non-ribosomal RNA-containing sample according to [1] or [2], wherein the step (a) of splitting subunits of ribosomes and mRNAs is performed by using a chelating agent.
[4] The method for producing a non-ribosomal RNA-containing sample according to any one of [1] to [3], wherein the step (b) of removing subunits of ribosomes split in the step (a) is performed by ultrafiltration.
[5] A method for analyzing a non-ribosomal RNA, which comprises the step of obtaining a non-ribosomal RNA-containing sample by performing the method for producing a non-ribosomal RNA-containing sample according to any one of [1] to [4], and the step of sequencing RNAs in the non-ribosomal RNA-containing sample.
[6] A kit for use in performing the method for producing a non-ribosomal RNA-containing sample according to any one of [1] to [4], which comprises a reagent for splitting subunits of ribosomes and mRNAs, and a means for removing subunits of ribosomes.

### BRIEF DESCRIPTION OF THE DRAWINGS

FIG. 1 is a schematic diagram of the present invention. In the conventional purification method (up to the indication of Purification (standard method) in the diagram), a monosome fraction obtained by ultracentrifugation or gel filtration after RNase treatment is treated with a solution containing a protein-denaturing agent (e.g., phenol, guanidine isothiocyanate, etc.) to purify footprints. In the modified method of the present invention (indicated as Modified method in the diagram), the monosome fraction obtained by ultracentrifugation or gel filtration is treated with a chelating agent to split ribosomes into large and small subunits and footprints (ribosome splitting with chelating agent), followed by purification of footprints by removal of the large and small subunits (removal of ribosome subunits).

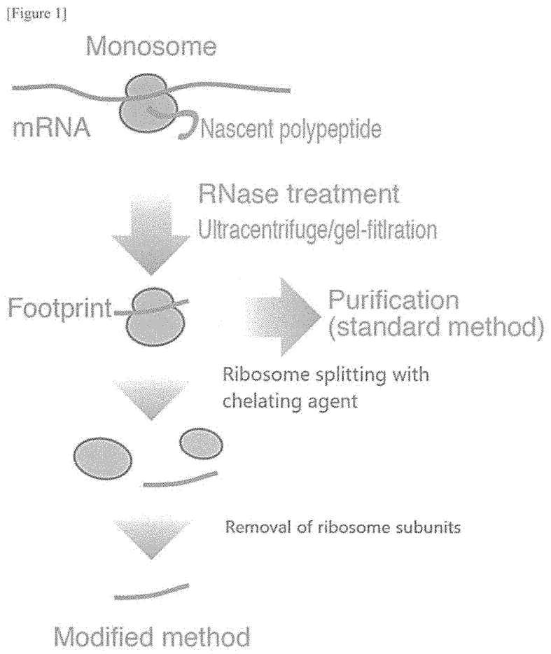

FIG. 2 shows numbers of reads obtained from the analyses of libraries prepared by the standard method, ribosome splitting method, standard method—rRNA depletion, and ribosome splitting method+rRNA depletion described in the examples. The term “Mapped” means number of reads mapped on protein coding regions (CDS), which reflects to number of ribosomes on mRNA. RPM means reads per million.

FIG. 3 shows Pearson's correlation coefficients for the numbers of reads obtained by the analyses of libraries prepared by the standard method, ribosome splitting method, standard method—rRNA depletion, and ribosome splitting method—rRNA depletion described in the examples (each repeated twice).

### MODES FOR CARRYING OUT THE INVENTION

The present invention may be explained hereafter with reference to typical embodiments or specific examples thereof, but the present invention is not limited by such embodiments etc. In this description, numerical ranges expressed by using “to” means ranges including the numerical values mentioned before and after “to” as the smallest and largest values. The indication “nt” used with respect to the length of RNA means nucleotide.

RNA-seq (RNA sequencing) is a technique that allows genome-wide profiling of gene expression levels. In this description, RNA-seq includes total transcriptome sequencing (total RNA-seq), which can provide a comprehensive picture of transcriptional profile of cells at biological moments, as well as targeted RNA sequencing, which measures only target transcripts to analyze differential expression or allele-specific gene expression, sequencing of small non-coding RNA involved in transcription regulation and translation regulation (e.g., transfer RNA, snoRNA, snRNA etc.), and microRNA sequencing. snRNAs (small nuclear RNAs) are a class of small RNAs present in the nuclei of eukaryotic organisms, and together with other proteins, involved in various reaction processes such as RNA splicing and rRNA processing. snoRNAs (small nucleolar RNAs) are a group of small RNAs involved in chemical modifications (such as methylation and pseudouridylation) of rRNAs and other RNAs. MicroRNAs are classified as functional non-coding RNAs, and they are functional nucleic acids that are encoded on the genome, undergo a multistep generative process to ultimately result in microRNAs of 20 to 25 bases length, and are involved in the regulation of basic life phenomena such as cell development, differentiation, proliferation and cell death.

The ribosome profiling is a technique for determining a large number of sequences of parts of mRNA that have been bound by ribosomes and thereby determining a region of mRNA that was actively translated in the cell at a particular moment by taking advantage of the fact that when an mRNA molecule is degraded with an enzyme or other means, a portion of the mRNA bound by a ribosome is protected from degradation and remains.

The term “footprint” used in this description refers to a portion of mRNA that was protected from degradation by an enzyme or the like in ribosome profiling and has remained. The length of the footprint is about 40 nt or shorter, generally about 30 nt.

The term “read” or “sequence read” used in this description generally refers to a data sequence of A, T, C, and G bases determined for a DNA or RNA sample. A read or sequence read referred to in this description is, among other things, a sequence determined for a DNA fragment in a sequencing library prepared from a non-ribosomal RNA-containing sample obtained according to the present invention.

In this description, the term “non-coding RNA” is used as a generic term for RNAs that do not encode proteins, and examples of non-coding RNA include rRNA, transfer RNA (tRNA), mitochondria-derived ribosomal RNA (Mt-rRNA), mitochondria-derived transfer RNA (Mt-tRNA), chloroplast-derived ribosomal RNA, chloroplast-derived transfer RNA, snRNA, snoRNA, microRNA, and so forth.

In this description, the term “non-ribosomal RNA” is used as a generic term for RNAs other than ribosomal RNA (rRNA), and examples of non-ribosomal RNA include mRNA, transfer RNA (tRNA), mitochondria-derived transfer RNA (Mt-tRNA), chloroplast-derived transfer RNA, snRNA, snoRNA, microRNA, and so forth.

### <Method for Producing a Non-Ribosomal RNA-Containing Sample>

The method for producing a non-ribosomal RNA-containing sample of the present invention comprises the step (a) of splitting subunits of ribosomes and mRNAs in a sample containing mRNAs and ribosomes, and the step (b) of removing the subunits of ribosomes split in the step (a).

Non-patent document 1 reported that 16% of sequence reads obtained by ribosome profiling were mapped on CDS, while most of the rest were derived front rRNA. Non-patent document 2 reported that 90.2% of the sequence reads obtained from an RNA-seq library prepared from total RNA in which mRNAs were not concentrated were derived from rRNAs. In the examples mentioned herein later, it was demonstrated that 92% of all reads obtained by the conventional standard method were derived from rRNAs. In order to reduce the number of rRNA-derived sequence reads, rRNA-subtraction oligonucleotides. which can hybridize to rRNA and trap it on magnetic beads, are used (Non-patent documents 2 and 3). In the examples mentioned herein later. it was demonstrated that even with this method. 77% of the total reads were derived from rRNA, and reads derived from mRNA accounted for 18%.

The examples mentioned herein later demonstrated that, when a library for ribosomal profiling prepared from a non-ribosomal RNA-containing sample produced according to the present invention was analyzed. 23% of the total reads were derived from mRNA, and when the method using rRNA-subtraction oligonucleotides was used in combination, mRNA reads were increased to 50% of the total reads. According to the present invention, a non-ribosomal RNA-containing sample with a reduced percentage of rRNA content can be produced. Therefore, if a library for ribosome profiling or RNA-seq is prepared from a non-ribosomal RNA-containing sample produced according to the present invention, reads of mRNA and non-coding RNA, especially small non-coding RNA and microRNA, can be efficiently obtained. Use of the non-ribosomal RNA-containing samples produced according to the present invention is not limited to use in ribosome profiling, but are also effective for RNA-seq.

<Sample Containing mRNAs and Ribosomes>

The “sample containing mRNAs and ribosomes” referred to in the present invention means a crude extract of cells or tissues obtained by lysing or disrupting single cell, cell population, cultured cell or tissue containing mRNAs and ribosomes (henceforth referred to as lysate), and the single cell, cell population, cultured cell or tissue can be derived from any organism. Specifically, examples of the lysate include lysates of bacteria, fungi, animal cells or tissues, plant cells or tissues, and cultured cells thereof, but are not limited to these. The lysate can be prepared by cell lysis using a surfactant or physical disruption (e.g., mechanical disruption, homogenization in solution, sonication, freeze-thawing, disruption with mortar and pestle, etc.), and the preparation method can be appropriately selected according to the organism species, or cell or tissue type. The method for producing a non-ribosomal RNA-containing sample of the present invention can comprise the step of lysing or disrupting cells to obtain a lysate as a pretreatment.

The lysate is preferably prepared a by gentle means such as cell lysis using a surfactant to avoid degradation or damage of ribosomes. For the same reason, the lysate is preferably prepared without using any protein denaturing agent. Mg²⁻ chelating agent, or organic solvent such as phenol or chloroform. DNAs may be degraded by using, for example, DNase, since they interfere with the subsequent cDNA synthesis. Furthermore, the lysate can be obtained in the presence of the protein translation inhibitor, cycloheximide. In one embodiment, the lysate can be obtained as a supernatant obtained by suspending cells in a buffer containing a surfactant and cycloheximide, incubating them in the presence of DNase I (RNase-free), and centrifuging the cell suspension, as described in the examples mentioned later.

The sample containing mRNAs and ribosomes can be called a sample containing mRNAs and ribosomes as well as non-coding RNAs such as rRNA, transfer RNA (tRNA), mitochondria-derived ribosomal RNA (Mt-rRNA), mitochondria-derived transfer RNA (Mt-tRNA), chloroplast-derived ribosomal RNA, chloroplast-derived transfer RNA, snRNA, snoRNA, microRNA, and so forth.

### <Non-Ribosomal RNA-Containing Sample>

The “non-ribosomal RNA-containing sample” of the present invention can be obtained by performing the step (a) of splitting subunits of ribosomes and mRNAs in a sample containing mRNAs and ribosomes, and the step (b) of removing the subunits of ribosomes split in the step (a). The non-ribosomal RNA-containing sample may contain mRNA, transfer RNA (tRNA), mitochondria-derived transfer RNA (Mt-tRNA), chloroplast-derived transfer RNA, snRNA, snoRNA, microRNA, and so forth, and may also be concentrated for specific RNA species. The non-ribosomal RNA-containing sample may be a sample for preparing a sequencing library, and the sequencing library may be, for example, but not limited to, a library for ribosome profiling or RNA-seq. The rRNA content in the non-ribosomal RNA-containing sample of the present invention is reduced compared with the same in non-ribosomal RNA-containing samples obtained by conventional methods (e.g., the standard method described in Example 1). The ratio of the number of rRNA reads determined in a sequencing library prepared from a non-ribosomal RNA-containing sample of the present invention to the total number of reads is reduced compared with the same ratios of sequencing libraries prepared from samples obtained by conventional methods (e.g., the standard method described in Example 1).

<Step (a) of Splitting mRNAs and Subunits of Ribosomes>

Ribosome is a giant RNA-protein complex consisting of several rRNA molecules and about 50 different kinds of proteins, and the whole thereof consists of two particles, one large and one small. In this description, the two particles of the ribosome are referred to as the large subunit and the small subunit. As for the specific constitution of the ribosome, for example, in prokaryotic ribosomes, the large and small subunits are called 50S and 30S subunits, respectively, the 50S subunit consists of 23S rRNA (2904 nt), 5S rRNA (120 nt), and 34 different kinds of proteins, and has a molecular weight of 1600,000, and the 30S subunit consists of 16S rRNA (1542 nt) and 21 different kinds of proteins, and has a molecular weight of 900,000. The aggregate of both of these subunits constitutes the 70S particle, which has a molecular weight of 2,700,000. In eukaryotic ribosomes, the large and small subunits are called 60S and 40S subunits, respectively, the 60S subunit consists of 28S rRNA (4718 nt), 5.8S rRNA (160 nt). 5S rRNA (120 nt) and 50 different kinds of proteins, and has a molecular weight of 3,000,000. and the 40S subunit consists of 18S rRNA (1874 nt) and 33 different kinds of proteins, and has a molecular weight of 1,500,000. The aggregate of both of these subunits constitutes the 80S particle.

The ribosome holds mRNA, and serves as a site where genetic information of mRNA is read and translated into a protein. By splitting the aggregate of the two subunits into the small and large subunits, the mRNA held by the ribosome can be separated from the ribosome.

The step (a) of splitting mRNAs and subunits of ribosomes can be carried out by any method, for example, by removing Mg²⁻ ions, which are necessary to maintain the association of the both subunits. Mg²⁻ ions can be removed by any method, for example, by using a chelating agent. In other words, the step (a) of splitting mRNAs and subunits of ribosomes can be performed by using a chelating agent. Examples of the chelating agent include, for example, ethylenediaminetetraacetic acid (EDTA), nitrilotriacetic acid (NTA), diethylenetriaminepentaaeetic acid (DTPA), glycol ether diaminetetraacetic acid (EGTA, GFDTA), and so forth, and ethylenediaminetetraacetic acid (EDTA) is particularly preferred. The concentration of the chelating agent can be, in the case of ethylenediaminetetraacetic acid (EDTA), from 0.1 to 30 mM, preferably 5 to 15 mM. The treatment with the chelating agent can be performed by, in the case of EDTA, placing the reaction vessel on ice for 30 seconds to 60 minutes, and the treatment time can be appropriately changed.

<Step (b) of Removing Subunits of Ribosomes Split in Step (a)>

The step (b) of removing the subunits of ribosomes split in the step (a) is the step of removing the large and small subunits split in the aforementioned step (a) of splitting subunits of ribosomes and mRNAs from the sample containing mRNAs and ribosomes. The removal of the large and small subunits can be performed by employing any method that can remove them using difference of their sizes, for example, ultrafiltration, size exclusion chromatography (SEC), and so forth. As described later, the molecular weight of the small subunit of prokaryotes, the smallest of the subunits of ribosome, is approximately 900,000. Since transfer RNA (tRNA), mitochondria-derived transfer RNA (Mt-tRNA), chloroplast-derived transfer RNA, snRNA, snoRNA, microRNA, and so forth are sufficiently smaller than the small subunit of prokaryotes, the smallest among the subunits of ribosome, they can be separated from the large and small subunits on the basis of the differences of the sizes. On the other hand, since mRNAs have various lengths, and in the case of humans, many of them include more than 1000 nucleotides, they may not be separated from the large and small subunits using the size differences. Therefore, it is preferable to perform the step of fragmenting RNAs described below before “the step (b) of removing the subunits of ribosomes split in the step (a)” to fragment the mRNAs to a size that allows separation from the large and small subunits.

Ultrafiltration is a method of concentrating or removing components from a solution by passing the solution through a membrane. Ultrafiltration membranes have a molecular weight cut off (MWCO), molecules of a molecular weight larger than the MWCO of the membranes are retained on the membranes, and such molecules of a molecular weight larger than the MWCO of the membranes are removed from the permeate. In the present invention, the step of removing the large and small subunits of ribosomes can be performed by ultrafiltration. More specifically, by using ultrafiltration, RNAs contained in a sample containing mRNAs and ribosomes can be passed through a membrane to retain the large and small subunits of ribosomes on the membrane, and thereby a permeate containing RNAs can be obtained. The RNA recovered by the permeation through the ultrafiltration membrane may be any RNA, such as mRNA, tRNA, Mt-tRNA, chloroplast-derived transfer RNA, snRNA, snoRNA, and microRNA, and may also include rRNA.

As the ultrafiltration membrane, an ultrafiltration membrane that can permeate RNAs and retain the large and small subunits of ribosomes on the membrane can be used. The permeability and retention property of ultrafiltration membranes vary depending on various conditions, such as filtration pressure, presence of other solutes, molecular shape, adsorption property, and ionic strength. Therefore, although examples of selectable ultrafiltration membrane will be shown below, it is not limited to them, and the optimal one can be appropriately selected in consideration of RNA recovery rate and filtration speed.

Ultrafiltration membranes that can be used in the step (b) of the method for producing a non-ribosomal RNA-containing sample of the present invention can be selected from those having a molecular weight cut off (MWCO) in the range of 10 K (henceforth K represents 10³) to 2000 K, 10 K 1500 K, 10 K to 1000 K, 10 K to 900 K, 10 K to 800 K, 10 K to 700 K, 10 K to 000 K, 10 K to 500 K, 10 K to 400 K, 10 K to 300 K, 10 K to 100 K, 30 K to 2000 K, 30 K to 1500 K, 30 K to 1000 K, 30 K to 900 K, 30 K to 800 K, 30 K to 700 K, 30 K to 600 K. 30 K to 500 K, 30 K to 400 K, 30 K to 300 K, or 30 K to 100 K, but are not limited to these.

More precisely, as for the removal of the large and small subunits with a ultrafiltration membrane, since the molecular weight of the small subunit of prokaryotes, the smallest one among the subunits of ribosomes, is about 900,000, if a ultrafiltration membrane having a molecular weight cut off (MWCO) smaller than 900 K, preferably an MWCO of 150 K to 300 K, is used, the large and small subunits of prokaryotes can be retained on the membrane and removed. Further, since the molecular weight of the small subunit of eukaryotes is about 1,500,000, if a ultrafiltration membrane having an MWCO smaller than 1500 K, preferably an MWCO of 250 K to 500 K, is used, the large and small subunits of eukaryotes can be retained on the membrane and removed. From the viewpoint of retention ratio of the large and small subunits on the membrane, it is preferable to use a membrane having an MWCO smaller than 500 K, more preferably a membrane having an MWCO smaller than 300 K.

The method for producing a non-ribosomal RNA-containing sample of the present invention may comprises the step of degrading or fragmenting RNAs, as described later. When the method comprises such a step, as a result of the RNA degradation, RNAs remain as footprints of 40 nt length or smaller, or fragmented into a length appropriate for the sequencing platform. Selection of ultrafiltration membrane for recovery of RNA fragments based on the molecular weight of RNA (approximately 320.5/base) will be described below. For example, when a membrane having an MWCO in the range of 30 K to 500 K is used, RNAs of about 40 nt or shorter (molecular weight of about 13,000 or smaller) can be passed through, and the large and small subunits of prokaryotes and eukaryotes can be retained on the membrane. If an ultrafiltration membrane having an MWCO in the range of 100 K to 500 K is used. RNAs of about 100 nt or shorter (molecular weight of about 32,000 or smaller) can be passed through, and the large and small subunits of prokaryotes and eukaryotes can be retained on the membrane. If an ultrafiltration membrane having an MWCO of 300 K to 500 K is used. RNAs of about 500 nt or shorter (molecular weight of about 160,000 or smaller) can be passed through, and the large and small subunits of prokaryotes and eukaryotes can be retained on the membrane. If an ultrafiltration membrane having an MWCO of 500 K is used, RNAs of about 1,000 nt or shorter (molecular weight of about 320,000 or smaller) can be passed through, and the large and small subunits of prokaryotes and eukaryotes can be retained on the membrane. It is believed that such linear molecules as RNA can pass through a membrane that can block spherical molecules of the same molecular weight.

In one embodiment, the ultrafiltration can be performed by using a centrifugal ultrafiltration filter unit consisting of a tube equipped with an ultrafiltration membrane. Such a centrifugal ultrafiltration filter unit can be further inserted into a tube to constitute a double-layered centrifugal ultrafiltration tube. The centrifugal ultrafiltration filter unit can be used in accordance with the supplier's instructions for use.

The step (b) of removing the subunits of ribosomes split in the step (a) can be performed by size exclusion chromatography. In one embodiment, the step (b) of removing the subunits of ribosomes split in the step (a) can be performed by gel filtration chromatography using a spin column. Specifically, it can be performed according to, for example, the following procedure using Illustra™ MicroSpin™ S-400 HR Column (GE Healthcare, cat. no. 27-5140-01): 1. Mix the content of S-400 Column well, take out and place the tip end of the column into a 2 mL reservoir tube. 2. Load 700 μl of a lysis solution (20 mM Tris-HCl (pH 7.5), 150 mM NaCl, 5 mM EDTA, 1 mM dithiothreitol (DTT), 100 μg/ml cyclohexamide, RNase-free water, placed on ice) on the S-400 Column, and centrifuge it at 740 g and 4° C. for 1 minute. Place the column into a new 2 mL reservoir tube. Repeat this procedure three times in total. 3. Place the S-400 column into a new clean 1.5 mL tube. Load 100 μL of the solution obtained by performing the step (a) of “splitting subunits of ribosomed and mRNAs” mentioned above onto the center of the column resin, and centrifuge the column at 740 g and 4° C. for 2 minutes. 4. Load additional 100 μL of the lysis solution, and centrifuge the column at 740 g and 4° C. for 2 minutes to obtain an eluate.

In another embodiment, the step (b) of removing the subunits of ribosomes split in the step (a) can be performed by site exclusion chromatography using ultra high pressure liquid chromatography (uHPLC) (Yoshikawa et al., eLife 2018:7:c36530 DOI: 10.7554.eLife.36530). Specifically, a 7.8×300 mm column containing 5 μm particles, e.g., Thermo BioBasic SEC 300A, 1,000A, and 2,000A columns, or Agilent Bio SEC-5 2,000A Column can be used. By using Dionex Ultimate 3,000 Bio-RS uHPLC system (Thermo Fisher Scientific), each SEC column is equilibrated with two column volumes (CV) of filtered SEC buffer (20 mM Hepes-NaOH (pH 7.4), 60 mM NaCl, 30 mM EDTA, 0.3% CHAPS, 0.2 mg/mL heparin, 2.5 mM DTT), 100 μL of a 10 mg/mL filtered bovine serum albumin (BSA) solution diluted with PBS is injected once to block the sites of nonspecific interaction. 10 μL of a 10 mg/mL BSA solution and 25 μL of a standard solution containing HyperLadder 1 kb (BIOLINE) are injected, the condition of the column is monitored, and then the solution obtained by carrying out “the step (a) of splitting the subunits of ribosomes and mRNAs” described above is injected into the column. The chromatogram is monitored by measuring UV absorbance at 215, 260, and 280 nm at a data collection rate of 1 Hz with a diode array detector. The flow rate is 0.8 mL/minute, and 48×100 μL fractions, 24×200 μL fractions, or 16×300 μL fractions are collected at 4° C. in 9 to 14.6 minutes by using 1 mL low protein-binding 96-deep well plate Eppendorf). Peaks are quantified by using Chromeleon 6.8 Chromatography Data System (Thermo Fisher Scientific).

After the subunits of ribosomes have been removed by ultrafiltration, size exclusion chromatography, or the like, the sample can be subjected to purification. Purification can be performed by using known RNA purification methods, for example, by using a solution containing phenol:chloroform, phenol and guanidine isothiocyanate, or the like.

The method for producing a non-ribosomal RNA-containing sample of the present invention can be combined with known rRNA removal methods, monosome concentration methods, and mRNA concentration methods. The known rRNA removal methods that can be combined include the method using rRNA-subtraction oligonucleotides that can hybridize to rRNAs and trap them on magnetic beads (Non-patent documents 2 and 3), and it can be performed by using Ribo-Zero (registered trademark) rRNA Removal Kit (Illumina). The known mRNA concentration methods that can be combined include the polyA selection method, in which polyA-tailed RNA can be concentrated by using Oligotex (registered trademark)-dT30 <Super> mRNA Purification Kit (Takara) or oligo(dT)-Dynabeads (registered trademark). The known methods for monosome concentration that can be combined include sucrose density gradient centrifugation, sucrose cushion centrifugation, gel filtration chromatography using the spin column mentioned above, and so forth.

If a sequencing library is prepared from a non-ribosomal RNA-containing sample produced according to the present invention, the number of rRNA reads relative to the total number of reads can be reduced. Specifically, the ratio of the number of rRNA reads to the total number of reads can be reduced to 80% or less, 75% or less, 70% or less, 65% or less, 60% or less, 55% or less, 50% or less, 45% or less, 45% or less, 40% or less, 35% or less, 30% or less, 25% or less, 20% or less, 15% or less, or 10% or less. Furthermore, the number of rRNA reads relative to the total number of reads can further be reduced by combining the production method of the present invention with the method using rRNA-subtraction oligonucleotides that can hybridize to rRNA and trap it on magnetic beads (Non-patent documents 2 and 3). Specifically, the ratio of the number of rRNA reads to the total number of reads can be reduced to 50% or less, 45% or less, 40% or less, 35% or less, 30% or less, 25% or less, 20% or less, 15% or less, or 10% or less.

### <Step of Degrading or Fragmenting RNAs>

The method for producing a non-ribosomal RNA-containing sample of the present invention can further comprise the step of degrading or fragmenting RNAs in the sample containing mRNAs and ribosomes. Ribosome profiling usually comprises the step of degrading RNAs, and in the present invention, the step of degrading RNAs can be performed before the step (a) of splitting subunits of ribosomes and mRNAs. If RNA to be analyzed contained in a non-ribosomal RNA-containing sample is long, and cannot be separated from the large and small subunits using difference of the sizes, the fragmentation of mRNA can be performed before “the step (b) of removing the subunits of ribosomes split in the step (a)”.

The step of degrading RNAs can be performed for the purpose of, for example, obtaining footprints to be analyzed by ribosome profiling. The step of degrading RNAs can be performed by, for example, enzymatic degradation. Degradation of RNAs means modifying RNAs so that RNAs have a length shorter than that before the degradation, and enzymatic degradation of RNAs means degrading RNAs by using an enzyme that can modify RNAs so that RNAs have a length shorter than that before the degradation. The enzyme to be used can be a ribonuclease (RNA-degrading enzyme) such as endoribonuclease or exoribonuelease, and a single strand-specific RNA endonuclease such as RNase I can be used. Other enzymes that can be used for RNA degradation include RNase A, RNase T1, and so forth. RNA degradation may also be performed by partial alkaline hydrolysis as described later.

In one embodiment, in the method for producing a non-ribosomal RNA-containing sample for preparing a library for ribosome profiling, the step of degrading RNAs is performed prior to the step (a) of splitting subunits of ribosomes and mRNAs, and the RNA degradation can be performed by digestion with RNase 1. By the digestion with RNase 1, RNA molecules in the sample containing mRNAs and ribosomes are degraded, but parts of mRNAs to which ribosomes bind are protected from the degradation. The part of mRNA to which one ribosome binds is called monosome. Monosomes in a sample containing mRNAs and ribosomes can be concentrated by sucrose density gradient centrifugation, sucrose cushion centrifugation, gel filtration chromatography using the spin column mentioned above, or the like.

The step of fragmenting RNAs can be performed, for example, in the step (b) of removing the subunits of ribosomes split in the above step (a) for the purpose of making RNAs sufficiently small so that subunits of ribosomes can be separated by using difference of the sizes, and RNAs can be made to have a desired size by any means that can achieve the purpose.

The step of fragmenting RNAs can be performed by, for example, enzymatic fragmentation, chemical fragmentation, and/or mechanical fragmentation. RNA fragmentation means cut RNAs into appropriate fragment sizes, for example, a length suitable for the sequencing platform. In one embodiment, the RNA fragmentation can be performed by partial alkaline hydrolysis. The partial alkaline hydrolysis can be performed by, for example, adding 10 μL of 2× alkaline hydrolysis solution (2 mM EDTA, 12 mM Na.CO₃, 88 mM NaHCO₃, pH 0.3) to an equal volume of an RNA-containing solution (e.g., the lysate), mixing them, treating the mixture at 95° C. for 20 minutes, placing it on ice after the treatment, and adding 300 μL of 0.3 M NaOAc (pH 5.2). The fragmentation by partial alkaline hydrolysis can be performed in a highly controlled manner so that RNAs are degraded into an appropriate size, and for the present invention, the fragmentation is performed so that RNAs are degraded to have a size of, for example, 100 to 3000 nt, preferably 100 to 1000 nt, more preferably 100 to 500 nt, even more preferably 100 to 300 nt.

In one embodiment, the RNA fragmentation can be performed by ultrasonic shearing. Ultrasonic shearing can be performed by, for example, placing the lysate in a 15-mL Falcon tube and subjecting it to ultrasonication at 4° C. on a water bath using an existing ultrasonic disruption machine. The fragmentation by ultrasonication can be performed in a highly controlled manner so that RNAs have an appropriate size, and for the present invention, RNAs are fragmented to have a size of, for example, 100 to 3000 nt, preferably 100 to 1000 nt, more preferably 100 to 500 nt, even more preferably 100 to 300 nt.

In one embodiment, the RNA fragmentation can be enzymatically performed. For example, it is preferable to use an enzyme that can randomly fragment single-stranded RNAs to a desired size in a nucleotide sequence-independent manner without any bias. Specifically, RNase I, RNase A, RNase T1, RNase T2, MNase (Micrococcal Nuclease), RNase V1, RNase S1, and so forth can be used. Enzymatic fragmentation can be performed in a highly controlled manner so that RNAs have an appropriate size, and for the present invention, RNAs are fragmented to have a size of, for example, 100 to 3000 nt, preferably 100 to 1000 nt, more preferably 100 to 500 nt, even more preferably 100 to 300 nt.

In the method for producing a non-ribosomal RNA-containing sample for preparing a library for RNA-seq, either the step of fragmenting RNAs or the step (a) of splitting ribosomal subunits and mRNAs can be performed first so long as the ribosomes are not broken.

In yet another embodiment, a non-ribosomal RNA-containing sample may be produced by implementing the method for producing a non-ribosomal RNA-containing sample without performing the step of degrading or fragmenting RNAs. For example, this is such a case that a library for RNA-seq is prepared for analysis of small non-coding RNAs (e.g., tRNA, snoRNA, snRNA etc.) and microRNAs. This is because small non-coding RNAs are as short as approximately 200 nt or shorter, microRNAs are as short as 30 nt or shorter, and therefore they can be separated from the subunits of ribosomes on the basis of the size difference without fragmentation.

### <Method for Analyzing Non-Ribosomal RNA>

The method for analyzing non-ribosomal RNA of the present invention comprises the step of obtaining a non-ribosomal RNA-containing sample by implementing the method for producing a non-ribosomal RNA-containing sample mentioned above, and the step of determining nucleotide sequence of RNA in the non-ribosomal RNA-containing sample. The non-ribosomal RNA includes mRNA (including footprint), tRNA, Mt-tRNA, chloroplast-derived transfer RNA, snRNA, snoRNA, microRNA, and so forth. More specifically, the method for analyzing non-ribosomal RNA means RNA-seq (RNA sequencing) method or ribosome profiling method.

The step of determining nucleotide sequence of RNA in the non-ribosomal RNA-containing sample is the step of determining nucleotide sequence of RNA using a non-ribosomal RNA-containing sample obtained by performing the method for producing a non-ribosomal RNA-containing sample. The determination of nucleotide sequence includes determination of bases constituting RNA as well as determination of chemical modification in bases constituting RNA. RNA sequencing may be performed by using an amplification product contained in the sequencing library prepared from the non-ribosomal RNA-containing sample.

The method for preparing a sequencing library typically comprises the step of reverse transcription into cDNA using a reverse transcriptase and amplifying the resulting reverse transcription product by using an appropriate nucleic acid amplification method. The term “amplifying (a nucleic acid)” refers to a process of subjecting a nucleic acid to at least one round of elongation, replication or transcription for the purpose of increasing (e.g., exponentially increasing) the copy number of the nucleic acid. The copy of the nucleic acid may be a complementary copy of the nucleic acid. It is also more preferred that multiple rounds of elongation, replication or transcription are performed in this step. The nucleic acid amplification method is not particularly limited, and examples include, for example, PCR amplification, rolling circle amplification, and so forth. For the method for preparing a sequencing library, the descriptions of Patent document 1, Non-patent documents 1 to 3 mentioned above, and so forth can also be referred to as required.

In one embodiment, for the preparation of a sequencing library for ribosome profiling, a non-ribosomal RNA-containing sample selectively containing footprints can be obtained by performing the method for producing a non-ribosomal RNA-containing sample of the present invention. For example, in a sample containing mRNAs and ribosomes (a sample prepared by obtaining a lysate in the presence of cycloheximide, degrading RNAs in the lysate with RNase I, and subjecting the lysate to sucrose cushion centrifugation to enrich monosomes), the method for producing a non-ribosomal RNA-containing sample comprising the step (a) of splitting subunits of ribosomes and mRNAs, and the step (b) of removing the subunits of ribosomes split in the step (a) is performed to obtain a non-ribosomal RNA-containing sample. A non-ribosomal RNA-containing sample that selectively contains footprints at the time when the lysate was prepared can be thereby obtained. The preparation of a sequencing library for ribosome profiling can be performed as described in the examples mentioned later, that is, such a library can be produced by subjecting the non-ribosomal RNA-containing sample to denaturing polyacrylamide gel electrophoresis together with RNA size markers, cutting bands of RNAs of 26 nt to 34 nt length out from the gel, purifying RNAs from the gel, adding linkers to them, reverse-transcribing them into cDNAs, cyclizing them, amplifying them by PCR, and adding barcodes to them according to the method of the examples described later. After bands of RNAs of desired lengths are cut out, and RNAs are purified from the gel, the addition of linkers, adapters. or barcodes, reverse transcription, and PCR amplification can be performed according to any known methods, preferably any known methods used for analysis on a next-generation sequencer.

In one embodiment, a non-ribosomal RNA-containing sample selectively containing mRNAs can be obtained by performing the method for producing a non-ribosomal RNA-containing sample of the present invention for the preparation of a sequencing library for total transcriptome sequencing (total RNA-seq). In a preferred embodiment, the method for producing a non-ribosomal RNA-containing sample comprising the step (a) of splitting subunits of ribosomes and mRNAs, and the step (b) of removing the subunits of ribosomes split in the step (a) is performed in a sample containing mRNAs and ribosomes (a sample prepared by obtaining a lysate in the presence of cycloheximide, and subjecting it to poly-A selection for mRNAs, and fragmentation of RNAs) to obtain a non-ribosomal RNA-containing sample. A non-ribosomal RNA-containing sample containing fragments of mRNAs expressed at the time when the lysate was prepared can be thereby obtained. A sequencing library for total RNA-seq can be produced by subjecting the non-ribosomal RNA-containing sample to denaturing polyacrylamide gel electrophoresis together with RNA size markers, cutting bands of RNAs of a length suitable for the sequencing platform, such as 26 to 500 nt length, out from the gel, purifying RNAs from the gel, adding linkers to them, reverse-transcribing them into cDNAs, cyclizing them, amplifying them by PCR, and adding barcodes to them according to the methods of the examples described later. After bands of RNAs of desired lengths are cut out, and RNAs are purified from the gel, the addition of linkers, adapters, or barcodes, reverse transcription, and PCR amplification can be performed according to any known methods, preferably any known methods used for analysis on a next-generation sequencer.

In one embodiment, for the preparation of a sequencing library for analysis of small molecule RNA such as tRNA, snRNA, and snoRNA or analysis of microRNA, a non-ribosomal RNA-containing sample containing tRNA, snRNA, snoRNA, and microRNA can be obtained by performing the method for producing a non-ribosomal RNA-containing sample of the present invention. In a preferred embodiment, the method for producing a non-ribosomal RNA-containing sample comprising the step (a) of splitting subunits of ribosomes and mRNAs, and the step (b) of removing the subunits of the ribosomes split in the step (a) is performed in a sample containing mRNAs and ribosomes to obtain a non-ribosomal RNA-containing sample. For example, a sequencing library for microRNA analysis can be produced by subjecting a non-ribosomal RNA-containing sample to denaturing polyacrylamide gel electrophoresis together with RNA size markers, cutting bands of RNAs of 18 to 30 nt length out from the gel, purifying RNAs from the gel, adding linkers to them, reverse-transcribing them into cDNAs, cyclizing them, amplifying them by PCR, and adding barcodes to them according to the method of the examples described later. After bands of RNAs of desired lengths are cut out, and RNAs are purified from the gel, the addition of linkers, adapters, and barcodes, reverse transcription, and PCR amplification can be performed according to any known methods, preferably any known methods used for analysis on a next-generation sequencer.

As the sequencing technique for the sequencing, method using a next-generation sequencer can be used. The type of the next-generation sequencer is not particularly limited, and examples thereof include HiSeq2000 (Illumina), Genome Analyzer IIx (Illumina), Genome Sequencer-FLX (Roche), and so forth. RNA sequencing using a next-generation sequencer comprises the step of immobilizing nucleic acids on a flow cell or microarray. In the sequencing process, bridge amplification (especially by bridge PCR) may occur in a flow cell or on a microarray, both of which immobilizes nucleic acids.

RNA sequencing is achieved by using the “sequencing by synthesis (SBS)” technique. The SBS technique used herein refers to a technique for sequencing a subject nucleic acid by synthesizing a complementary strand of the nucleic acid. The SBS technique may be selected from the group consisting of “pyrosequeneing”, “sequencing by ligation”, and “sequencing by extension”. The “pyrosequencing” refers to a method of sequencing by detecting pyrophosphate produced upon nucleotide incorporation. The “sequencing by ligation” refers to a method of nucleic acid sequencing using a ligase to identify nucleotides present at designated positions within a nucleic acid sequence. The “sequencing by extension” refers to a method of nucleic acid sequencing in which primers are extended with known or detectable nucleotides.

“Deep sequencing” can also be employed as the sequencing technique for performing the sequencing. The “deep sequencing” refers to a method of sequencing in which multiple nucleic acids are determined in parallel (Bentley et al., Nature. 2008. 456:53-59). In a typical sequencing protocol using “deep sequencing”, a nucleic acid (e.g., DNA fragment) is attached to the surface of a reaction platform (e.g., flow cell, microarray. etc.). The attached nucleic acid is amplified in situ, and can be used as template for synthetic sequencing (e.g., SBS) using a detectable label (e.g., fluorescent reversible terminator deoxyribonucleotide). Typical reversible terminator deoxyribonucleotides include 3′-O-azidomethyl-2′-deoxynucleoside triphosphates of adenine, cytosine, guanine, and thymine, each of which may further be labeled with a mutually recognizable and removable fluorophore via a linker. The sequencing may be performed by the single read method or pair-end method.

When various nucleotide sequences in a sequencing library are determined in the step of sequencing of RNAs contained in the non-ribosomal RNA-containing sample, the sequence reads are aligned to a reference sequence, and after the alignment, various analyses such as identification of single nucleotide polymorphism (SNP), insertion and deletion (indel), read counts for RNA analysis method, phylogenetic evolutionary analysis, and metagenomic analysis can be performed.

In the step of sequencing RNAs in a non-ribosomal RNA-containing sample, presence of sequence reads of rRNA that may contaminate is also revealed by alignment to a reference sequence.

### <Kit>

The kit used for performing the method for producing a non-ribosomal RNA-containing sample of the present invention includes a reagent for splitting the subunits of ribosomes and mRNAs, and a means for removing the subunits of ribosomes. The reagent for splitting the subunits of ribosomes and mRNAs may be a chelating agent, examples of the chelating agent include ethylenediaminetetraacetic acid (EDTA), nitrilotriacetic acid (NTA), diethylenetriaminepentaacetie acid (DTPA), glycol ether diaminetetraacetic acid (EGTA, GEDTA), and so forth, and ethylenediaminetetraacetic acid (EDTA) is particularly preferred. The means for removing subunits of ribosomes may be a centrifugal ultrafiltration filter unit comprising a tube equipped with an ultrafiltration membrane. Such a centrifugal ultrafiltration filter unit may also be further inserted into a tube to constitute a double-layered centrifugal ultrafiltration tube.

### EXAMPLES

The present invention will be more specifically explained with reference to the following examples. However, the present invention is not limited to these examples. In this description, unless especially stated, “%” and so forth are mass-based, and numerical ranges are mentioned so as to include their end points.

Materials and equipments used in the examples are listed at the end of the section of Examples.

### Example 1

### Preparation of Sequencing Libraries by the Standard Method

Libraries for ribosome profiling were prepared from HEK293 cells (AFCC. cat. no. CRL-1573) using the procedure described in McGinley N. J. et al., 2017 (Non-patent document 3). The protocol based on McGinley N. J. et al., 2017 (Non-patent document 3) is described below. In this description, the protocol described in Example 1 is referred to as the “standard method”.

### Section 1. Preparation of Lysates

### <Cycloheximide Treatment>

TABLE 1
Lysis Buffer

| | [x1 sample] | [In 5 mL] | [Final] |
|---|---|---|---|
| 1M Tris-HCl pH 7.5 | 12 | 100 | 20 mM |
| 5M NaCl | 18 | 150 | 150 mM |
| 1M MgCl₂ | 3 | 25 | 5 mM |
| 0.1M DTT | 6 | 50 | 1 mM |
| 10% Triton X-100 | 60 | 500 | 1% |
| RNase-free water | 500.4 | 4170 | |
| Total | 600 | 5 mL | |

Make the above buffer and let it cool.
Add the following reagent just before use.

TABLE 2

| | [In 5 mL] | [Final] |
|---|---|---|
| Cycloheximide 100 mg/mL | 5 | 100 microg/mL |

1. Rinse the cells on a 10 cm dish with 5 mL of cooled PBS.
2. Aspirate PBS thoroughly, and add 400 μL of the lysis buffer (EDTA-free), taking care to cover the entire surface of the dish.
3. Detach the cells by pipetting and transfer them into a DNA LoBind tube. Wash the dish again with 200 μL of the lysis buffer, and add the buffer to the tube.
4. Add 7.5 μL of Turbo DNase I (2 U/μL), and keep the mixture on ice for 10 minutes.
5. Centrifuge the mixture (lysate) at 20,000× g and 4° C. for 10 minutes.
6. Recover the supernatant in a tube and stir it by inversion.
7. Separate 5 μL of the lysate into one tube for concentration measurement, and then divide the rest in a volume of 100 μL each, flash freeze them with liquid nitrogen, and store at −80° C.
   <RNA Concentration Measurement with Qubit RNA BR Assay Kit>
1. Prepare Working Solution 200 μL×(2 tubes for standard—4 tubes for samples). Working Solution=Qubit RNA BR Reagent 1 μL:Qubit RNA BR Buffer 200 μL.
2. Add 190 μL of Working Solution for standard and 199 μL for the sample to 0.5 mL tubes. Add 10 μL of Standard Reagents #1 and #2, and 1 μL of sample to each tube, vortex the mixture, and spin down. Incubate the mixture for 2 minutes at room temperature.
3. Perform measurement with Qubit 2.0 Fluorometer, in which select RNA BR Assay. Perform measurement for standards and samples.
*80% confluent HEK cells yielded 200 to 300 ng/μL from one 10-cm dish (for 600 μL of lysate)

### Section 2. RNase Digestion to Ultracentrifugation (Sucrose Cushion)

Prepare a heat block at 25° C.
Cool a rotor or an ultracentrifuge.

TABLE 3
Sucrose Cushion (EDTA-free)

| | |
|---|---|
| [In 5 mL] | [Final] |

| Sucrose | 1.7 | g | 1M |
|---|---|---|---|
| 1M Tris-HCl pH 7.5 | 100 | | 20 mM |
| 5M NaCl | 150 | | 150 mM |
| 1M MgCl₂ | 25 | | 5 mM |
| 0.1M DTT | 50 | | 1 mM |
| RNase-free water | 3565 | | |
| Total | 5 | mL | |

Once the sucrose has dissolved, keep sucrose cushion on ice, and add the following just before use.

TABLE 4

| | [In 5 mL] | [Final] | |
|---|---|---|---|
| Cycloheximide 100 mg/mL | 5 | 100 | microg/mL |
| SUPERase In 20 U/microL | 5 | 20 | U/mL |

### <Sample Preparation>

Use Nase 1 at a concentration of 2 U/l1 ug RNA (use 20 U for 10 μg of RNA)

TABLE 5

| | [microL] |
|---|---|
| RNA Lysate (for RNA 10 microg) | X |
| Lysis buffer (prepared upon use) | Y |
| RNase 1 (10 U/micro L) | 2 |
| Total | 300 |

1. Take RNA in a tube, and add the lysis buffer to make 298 μL.
2. Add 2 μL of RNase I, stir the mixture gently, and incubate at 25° C. for 45 minutes on the heat block (be precise so that there is no difference in reaction time between samples).
3. Place the tube on ice, and immediately add 10 μL (200 U) of SUPERase In (RNase inhibitor) to the tube (move the tube onto ice first, as cooling helps to stop the reaction).
4. Transfer 300 μL of the sample after RNase I digestion to an ultracentrifuge tube.
5. Slowly pour 900 μL of Sucrose cushion buffer into the bottom of the sample to form two layers.
6. Centrifuge the layers at 100,000 rpm for 1 hour at 4° C. on a TLA110 rotor to obtain a ribosome pellet.
7. Aspirate the supernatant from the top of the liquid surface, taking care not to collapse the pellet, and discard the supernatant.

### Section 3. Footprint Fragment Purification

### <Direct RNA Recovery>

Add 300 μL of TRIzol reagent to the pellet (pellet becomes visible), dissolve the pellet well (referred as TRIzol sample), and transfer the solution to a DNA LoBind tube.

### <Direct-zol MicroPrep Kit Column Purification>

TABLE 6

| | [microL] |
|---|---|
| TRIzol sample | 300 |
| Ethanol (equal volume) | 300 |
| Total | 600 |

1. Transfer the solution to a column and centrifuge at 12,000× g and 4° C. for 1 minute. Discard the reservoir tube, and place the column into a new reservoir tube after every centrifugation.
2. Add 400 μL of PreWash buffer and centrifuge the mixture at 12,000× g and 4° C. for 1 minute. Repeat this operation twice.
3. Add 700 μL of Wash Buffer, and centrifuge the mixture at 12,000× g and 4° C. for 1 minute.
4. Perform empty centrifugation at 12,000× g and 4° C. for 5 minutes.
5. Add 6 μL of RNase-free water, and centrifuge the mixture at 12,000× g and for 2 minutes.
6. Add 6 μL of 2× RNA Loading Buffer.

### <RNA Size Marker Preparation and Electrophoresis>

TABLE 7

| For 2 lanes | [microL] |
|---|---|
| 10 microM N1800 (34 nt) | 1 |
| 10 microM N1801 (26 nt) | 1 |
| RNase-free water | 10 |
| 2x RNA Loading Buffer | 12 |
| Total | 24 |

1. Treat RNA size markers and samples on a heat block at 95° C. for 3 minutes and then on ice for 2 minutes. As the RNA size markers, Upper size marker oligoribonucleotide N1800 (34 nt) and Lower size marker oligoribonucleotide N1801 (26 nt) described in Non-patent document 3 (McGinley and Ingolia, 2017, Methods, 126, 112-129) were used.
2. Prepare a WAKO SuperSep RNA 15% gel.
3. After cleaning the wells, load the samples. Electrophorese them at 10 mA constant current for 50 minutes.
4. Stain the gel with 1× TBE 50 ml+SYBRGold 5 μL (10,000-fold dilution) for 3 minutes on a gentle shaker.
5. Place the gel on blue light, check the hands, and cut out the bands between 26 bp and 34 bp of the sample.
6. Cut out the bands of the markers as well.
   <RNA Extraction from Gel>

TABLE 8
<RNA Gel Extraction Buffer>

| | [microL] | [final] | |
|---|---|---|---|
| 3M Sodium acetate pH 5.2 | 400 | 300 | mM |
| 0.5M EDTA | 8 | 1 | mW |

| | | |
|---|---|---|
| 10% SDS | 100 | 0.25% |
| RNase-free waler | 3492 | |
| Total | 4000 | |

1. Crush the gel pieces in a 1.5 mL tube with a pestle.
2. Add 400 μL of the RNA gel extraction buffer, and wash off the gel on the pestle into the crushed gel.
3. Freeze the mixture at −80° C. for 30 minutes or in liquid nitrogen.
4. Stir the mixture by inversion at room temperature for 2 hours or longer.
5. Place the Spin-X column in a 1.5 mL tube of DNA Lobind, and transfer the gel solution into it with a thick tip.
6. Centrifuge the solution at 10,000× g and 4° C. for 1 minute.
7. Add 3 μL of GlycoBlue and 500 μL of isopropanol, and mix them well.
8. Place the mixture in a freezer for 1 hour, and then centrifuge at 20,000× g and 4° C. for 30 minutes.
9. Discard the supernatant, and rinse the pellet with 70 ethanol.
10. Add 7 μL of 10 mM Tris, pH 7.5 to the pellet and dissolve it.

### Section 4. Preparation of 20 μM Preadenylated Linker

1. Prepare the following solution in 8-strip tubes.

TABLE 9

| | [microL] |
|---|---|
| 100 microM 5′ p-linker-ddC primer | 1.2 |
| 10x 5′ DNA Adenylation reaction buffer | 2 |
| 1 mM ATP | 2 |
| Mtb RNA Ligase | 2 |
| RNase-free water | 12.8 |
| Total | 20 |

Treat the solution at 65° C. for 1 hour, and then at 85° C. for 5 minutes

As the 5′ p-linker-ddC primer mentioned in the above table, NI-810 to NI-817 described in Non-patent document 3 (McGinley and Ingolia, 2017. Methods. 126, 112-129, Table 8) were used.
2. Oligo Clean & Concentrator Column Purification

TABLE 10

| | [microL] |
|---|---|
| Sample 20 microL - RNase-free water 30 microL | 50 |
| Binding buffer (2-fold volume) | 100 |
| Total | 150 |
| Mix | + |
| Ethanol (8-fold volume) | 400 |
| Total | 550 |

Transfer the mixture to the column and centrifuge the column at 12,000× g for 1 minute. Add 750 μL of Wash buffer, and centrifuge the column at 12,000× g for 1 minute. Perform empty centrifugation at 12,000× g for 5 minutes. Perform elution with 6 μL of RNase-free water.

### PAUSE POINT −20° C.

### Section 5. Dephosphorylation and Linker Ligation

### <Dephosphorylation>

1. Prepare mixture
   Marker +4 samples

TABLE 11

| | [x1 sample] | [x5.2 (premix)] |
|---|---|---|
| RNA sample | 7 | — |
| 10x T4 PNK buffer | 1 | 5.2 |
| T4 PNK | 1 | 5.2 |
| SUPERase In | 1 | 5.2 |
| Total | 10 | 15.6 |

2. Transfer the samples to 8-strip tubes, treat them at 95° C. for 2 minutes, and then on ice for 3 minutes.
3. Add the mixture in a volume of 3 μL each, and incubate them at 37° C. for 1 hour.

### PAUSE POINT −80° C.

### <Linker Ligation>

Attach a different linker to each sample. Make a note of it.
Any linker can be attached to the marker.
1. After dephosphorylation, treat the sample at 95° C. for 2 minutes and then on ice for 3 minutes.

TABLE 12

| | [x1 sample] | [x5.2 (premix)] |
|---|---|---|
| 50% PEG-8000 | 7 | 36.4 |
| 10x T4 RNA ligase buffer | 1 | 5.2 |
| T4 Rnl2(tr)K227Q (200 U/microL) | 1 | 5.2 |
| Total | 9 | 46.8 |

2. Add 9 μL of the above mixture to each sample.
3. Add 1 μL of Preadenylated linker (20 μM), and mix them.
4. Incubate the mixture at 22° C. for 3 hours, and then at 4° C.
5. Perform Oligo Clean column purification (purify as described above).
6. Perform elution with 6 μL ×2× RNA Loading Buffer 6 μL.

### PAUSE POINT −80° C.

### <Preparation for Electrophoresis>

(1) Linker only

TABLE 13

| | [microL] |
|---|---|
| Preadenylated linker (20 microM) | 1 |
| RNase-free water | 5 |
| 2x RNA Loading Buffer | 6 |
| Total | 12 |

(2) Linker-ligated markers
(3) Linker-ligated samples
1. Treat sample or marker on a heat block at 95° C. for 3 minutes and then on ice for 2 minutes.
2. Flectrophorese each sample and markers as described above.
3. Cut out the bands of linker-ligated sample and markers.
*Since the samples contained in the gel portions have different linkers, they may be mixed thereafter.
4. Extract RNA from the gel (as described above)

### Section 6. Reverse Transcription Reaction

As the RT primer, the reverse transcription primer NI-802 described in Non-patent document 3 (McGliney and Ingolia. 2017, Methods, 126, 112-129) was used.
1. Prepare the followings in 8-strip tubes.
   (1) RT primer only (RNase-free water 10 μL).
   (2) RT primer+linker (RNase-free water 9.5 μL+20 μM linker 0.5 μL).
   (3) Marker (linker-ligated) 10 μL, or
   (4) Sample (linker-ligated) 10 μL
      plus
   1.25 μM RT primer NI802 2 μL
   Total 12 μL

Treat the sample at 6° C. for 5 minutes, and then on ice for 5 minutes.
2. Add 8 μL of a mixture having the following composition to each of (1) to (4)—RT primer.

TABLE 14

| | x1 | x4.2 (premix) |
|---|---|---|
| 5x Protoscript II buffer | 4 | 16.8 |
| 10 mM dNTPs | 1 | 4.2 |
| 10x DTT | 1 | 4.2 |
| SUPERase In | 1 | 4.2 |
| Protoscript II | 1 | 4.2 |
| (1) to (4) + RT primer | 12 | — |
| Total | 20 | 33.6 |

3. Incubate the resulting mixture at 50° C. for 30 minutes.
4. Add 2.2 μL of 1 M NaOH to the mixture and mix them. Treat the resulting mixture at 70° C. for 20 minutes.
5. Perform Oligo Clean & Concentrator Column purification

Add 28 μL of RNase-free water to make 50 μL. Perform purification as described above.

Perform elution with 6 μL+2× RNA loading buffer 6 μL.
PAUSE POINT −20° C.

### <Preparation for Electrophoresis>

When the linker and RT primer are annealed, they overlap with the sample in size. Therefore, treat the sample on a heat block at 100° C. for 5 minutes, and on ice, and then electrophorese it. DNA Gel Extraction Buffer (prepared during the electrophoresis).

TABLE 15

| | [microL] | [final] |
|---|---|---|
| 5M NaCl | 300 | 300 mM |
| 1M Tris pH 7.5 | 50 | 10 mM |
| 0.5M EDTA | 10 | 1 mM |
| RNase-free water | 4640 | — |
| Total | 5000 | — |

Cut out the gel portion of the sample, add DNA gel extraction buffer to the gel to extract DNA, subject the extract to isopropanol precipitation as described above, and dissolve the pellet in 12 μL of 10 mM Tris pH 7.5.

### Section 7. Circularization

1. Add the mixture in a volume of 8 μL each into 8-strip tubes.

TABLE 16

| | x1 sample | X4.2 (premix) |
|---|---|---|
| First strand cDNA | 12 | — |
| 10x CircLigase II buffer | 2 | 8.4 |
| 5M Betine | 4 | 16.8 |
| 50 mM MnCl2 | 1 | 4.2 |
| CircLigase II (100 U/microL) | 1 | 4.2 |
| Total | 20 | 33.6 |

2. Add 12 μL of sample
3. Treat the mixture at 60° C. for 1 hour, and then at 80° C. for 10 minutes.
PAUSE POINT -20° C.

### Section 8. PCR Amplification and Barcode Addition

Perform PCR for the sample with different numbers of cycles (6, 8, and 10 cycles), and cut out hands with few non-specific bands.

For the controls, (1) RT primer only, (2) RT primer-linker, and (3) marker linker-ligated), PCR may be performed only for 8 cycles.
1. For the sample, prepare 100 μL of reaction solution, and divide it into 3 wells in a volume of 33 μL each (for 6, 8 and 10 cycles).

For the control, prepare 100 μL of reaction solution without the template, divide it into 3 wells, and add 1.7 μL of the template to each well (8 cycles).

If the library is to be further pooled after PCR, use a different Rv primer for each library (NI822 to 826 described in Non-patent document 3 (McGinley and Ingolia, 2017. Methods, 126, 112-129)) as needed.

(Number of samples that can be pooled=Number of linker barcodes×Number of PCR primer barcodes)

TABLE 17

| | Sample | Control | 1 Well |
|---|---|---|---|
| 5x Phusion HF buffer | 20 | 20 | 6.7 |
| 2.5 mM dNTPs | 8 | 8 | 2.7 |
| 10 microM N1798 Fw primer | 5 | 5 | 1.7 |
| 10 microM N1799 Rv primer | 5 | 5 | 1.7 |
| Circularized cDNA template | 5 | — | 1.7 |
| H2O | 56 | 56 | 18.7 |
| Phusion polymerase (2 U/microL) | 1 | 1 | 0.3 |
| Total | 100 | 95 | 33 |

The NI798 Fw primer and NI799 Rv primer were the forward library PCR primer, NI-798 described in Non-patent document 3 (McGinley and lngolia, 2017, Methods. 126, 112-129), and Indexed reverse library PCR primer NI-799 mentioned in Table 9 of the same.
2. Once the PCR is started, collect the tubes at the end of the extension reaction of each cycle.
1) Allow the reaction at 98° C. for 30 seconds.
2) Repeat the cycle [98° C. for 10 seconds, 65° C. for 10 seconds, and 72° C. for 5 seconds] 6, 8, or 10 times (perform collection after every 2 cycles).
3) Allow the reaction at 72° C. for 5 minutes.
4) Place the reaction mixture at 4° C.
PAUSE POINT −20° C.
Section 9. Purification of PCR Product from Gel

The PCR product should not be denatured. As the gel, use Super Sep DNA. 15%.

As the loading dye, use 6× non-denaturing purple loading dye.

### <Preparation for Electrophoresis>

Mix 33 μL of sample—6× non-denaturing purple loading dye (6.5 μL), and electrophorese them in 2 wells in a volume of 19 μL each.

Mix RT primer, linker, and Marker 33 μL|6× non-denaturing purple loading dye 6.5 μL and electrophorese them in 2 wells in a volume of 10 μL each.

Perform electrophoresis at 20 mA for 1 hour and 20 minutes, and gel staining as described above.

Confirm the optimal cycle number, and cut out the hands.

<DNA Extraction from Gel>
1. Crush gel pieces for the 2 wells with a pestle, and add 230 μL of DNA gel extraction buffer.
2. After freezing the mixture at −80° C. or with liquid nitrogen, stir it by inversion at room temperature for at least 2 hours. Remove gel pieces with Spin-X column.
3. Perform NucleoSpin Gel and Clean column purification

TABLE 18

| | [microL] |
|---|---|
| DNA extract | 230 |
| Buffer NT1 (2-fold volume) | 460 |
| Total | 690 |

4. Transfer the sample to a column, and centrifuge it at 11,000× g and 4° C. for 1 minute.
5. Wash the column with 700 μL of Buffer NT3, and centrifuge it at 11,000× g and 4° C. for 1 minute. Repeat this operation twice.
6. Perform empty centrifugation at 11,000× g for 5 minutes.
7. Add 17 μL of NE buffer, and keep the mixture at room temperature for 1 minute
8. For elution, centrifuge the column at 11,000× g and 4° C. for 1 minute.
Section 10. Quality Check with MultiNA

### <Measurement in Ultra-Sensitive Mode

1/25 Gel Star (diluted 25 times with TE)

### <Ribosome Splitting and Ultrafiltration> was Performed.

### <Ribosome Splitting and Ultrafiltration>

1. Prepare a pellet suspension according to the following table, and place it on ice for about 5 minutes.

TABLE 21

| | Amount per run | | |
|---|---|---|---|
| Composition of pellet suspension | (μl) | Final in 1 ml | |
| 1M Tris-HCl pH 7.5 | 20 | 20 | mM |
| 5M NaCl | 60 | 300 | mM |
| 0.5M EDTA | 10 | 5 | mM |
| 0.1M DTT | 10 | 1 | mM |
| 10% Triton X-100 | 100 | 1% | |
| SUPERase In (20 U/μl) | 1 | 20 | U/ml |

| | | |
|---|---|---|
| RNase-free water | 799 | NA |

2. Place the ribosome pellet in a tube for ultrafiltration, and resuspend it in 150 μL of pellet suspension. EDTA in the pellet suspension splits the ribosomes into large subunits, small subunits, and footprints.
3. Transfer the mixture to a filter cup of AMICON® ULTRA 0.5 ML-100 KDa cutoff (Millipore, cat. no. UFC510024) equipped with an attached reservoir tube, and centrifuge it at 14,000× g and 4° C. for 10 minutes.
4. Discard the upper filter cup, and mix 360 μL of TRIzol LS reagent with the flow-through of the reservoir tube (referred to as TRIzol sample).
5. Perform the <Direct-zol MicroPrep Kit Column Purification> described in Example 1. Section 3. Footprint Fragment Purification. Then, perform experiments as described in Example 1. “4. Preparation of 20 μM Preadenylated Linker” to “10. Quality Check with MultiNA”.

### Example 3

Preparation of Sequencing Library by “Standard method—rRNA depletion”

In this example, samples were prepared by the “standard method” described in Example 1 plus an rRNA depletion step.
1/5 Shimadzu marker DNA-1000 (diluted 5 times with H₂O)
1/50 100 bp DNA ladder (diluted 50 times with TE)
1. Preparation of Separation Buffer

TABLE 19

| | [microL] |
|---|---|
| Separation buffer (DNA-1000) | 398 |
| 1/25 diluted Gel Star | 2 |
| Total | 400 |

2. Preparation of Sample and Ladder

TABLE 20

| | [microL] |
|---|---|
| Sample or 1/50 diluted ladder | 2 |
| 1/5 Shimadzu marker | 4 |
| Total | 6 |

3. Set the sample on the machine, and perform measurement.
4. Concentration and mol number are calculated as ⅕ of the measurement results (for ultra-sensitive measurement).

The volume required for sequencing is 15 μL for 1 nM solution.

If sufficient volume of sample is not available, perform PCR for 100 μL for only the optimal cycles, and purification from gel in the same manner.

### Example 2

### Preparation of Sequencing Library by Ribosome Splitting Method

In this example. a ribosome profiling library was prepared by splitting the subunits of ribosome:, and removing the ribosomes by ultrafiltration.

Lysates Were prepared from cells in the same manner as described in Example 1 (Example 1, Section I, Preparation of Lysates), and after RNase digestion, ultracentrifugation was performed to obtain a pellet of ribosomes (Example 1, Section 2, RNase Digestion to Ultracentrifugation (Sucrose Cushion)). In this Example 2, instead of <Direct RNA Recovery> described in Example 1, Section 3. Footprint Fragment Purification, the following <Ribosome Splitting and Ultrafiltration> was performed.

### <Ribosome Splitting and Ultrafiltration>

1. Prepare a pellet suspension according to the following table, and place it on ice for about 5 minutes.

TABLE 21

| | Amount per run | | |
|---|---|---|---|
| Composition of pellet suspension | (μl) | Final in 1 ml | |
| 1M Tris-HCl pH 7.5 | 20 | 20 | mM |
| 5M NaCl | 60 | 300 | mM |
| 0.5M EDTA | 10 | 5 | mM |
| 0.1M DTT | 10 | 1 | mM |
| 10% Triton X-100 | 100 | 1% | |
| SUPERase In (20 U/μl) | 1 | 20 | U/ml |

| | | |
|---|---|---|
| RNase-free water | 799 | NA |

2. Place the ribosome pellet in a tube for ultrafiltration. and resuspend it in 150 μL of pellet suspension. EDTA in the pellet suspension splits the ribosomes into large subunits, small subunits, and footprints.
3. Transfer the mixture to a filter cup of AMICON® ULTRA 0.5 ML-100 KDa cutoff (Millipore, cat. no. UFC510024) equipped with an attached reservoir tube, and centrifuge it at 14,000× g and 4° C. for 10 minutes.
4. Discard the upper filter cup, and mix 360 μL of TRIzol LS reagent with the flow-through of the reservoir tube (referred to as TRIzol sample).
5. Perform the <Direct-zol MicroPrep Kit Column Purification> described in Example 1, Section 3. Footprint Fragment Purification. Then, perform experiments as described in Example 1. “4. Preparation of 20 μM Preadenylated Linker” to “10. Quality Check with MultiNA”.

### Example 3

Preparation of Sequencing Library by “Standard method—rRNA depletion”

In this example, samples were prepared by the “standard method” described in Example 1 plus an rRNA depletion step.

Experiments were performed as described in Example 1, the sections of “1. Preparation of Lysates” to “5. Dephosphorylation and Linker Ligation”. Before “6. Reverse Transcription Reaction” described in Example 1. rRNA depletion was performed for the linker-ligated RNA sample as follows in “5. Dephosphorylation and Linker Ligation”.

<Ribosomal RNA Depletion (Ribo-Zero treatment)->

In Ribo-Zero treatment, 4 samples are processed together. Dissolve the sample in 10 mM Tris pH 7.5 to bring the total volume to 26 μL.
1. Transfer heads to a 225 μL tube, and stand them on a magnet.
2. Discard the supernatant, and perform washing twice with 225 μL of RNase-free water.
3. Suspend them in 60 μL of a resuspension solution. Place it at room temperature.
4. Prepare the following solution in 1.5 mL tubes.

TABLE 22

| | [microL] |
|---|---|
| RNA sample (for 4 samples) | 26 |
| Ribo-Zero reaction buffer | 4 |
| rRNA Removal SIn-Gold | 10 |
| Total | 40 |

Perform treatment at 68° C. for 10 minutes, and then at room temperature for 5 minutes.
5. Add 65 μL of the prepared beads, and perform pipeting and vortexing for 10 seconds. After leaving the mixture for 5 minutes at room temperature, perform vortexing for 10 seconds, and place it on a magnet.
6. Transfer the supernatant to a new tube.
7. Perform Oligo clean & Concentrator column purification.

TABLE 23

| | [microL] |
|---|---|
| Ribo-Zero-treated sample | 100 |
| Binding buffer (2-fold volume) | 200 |
| Total | 300 |
| Mix | + |
| Ethanol (8-fold volume) | 800 |
| Total | 1100 |

(Since the column can hold up to 800 μL, pass it through the column in two separate passes.)

Perform purification as described above, and perform elution with 10 μL of RNase-free water.

### Example 4

Preparation of Sequencing Library by “Ribosome Splitting Method rRNA Depletion”

In this example, samples were prepared by the “Ribosome splitting method” described in Example 2 plus an rRNA depletion step.

The experiments were performed as described in the sections “1. Lysate Preparation” through “5. Dephosphorylation and Liker Ligation” of Example 1, but <Ribosome Splitting and filtration> was performed instead of <Direct RNA Recovery> described in the section “3. Footprint Fragment Purification” of Example 1. Before “6. Reverse Transcription Reaction” of Example 1, rRNA depletion was performed for the linker ligated RNA sample in “5. Dephosphorylation and Linker Ligation” as described in Example 3, <Ribosomal RNA Depletion (Ribo-Zero treatment).

### Example 5

The libraries prepared and quality-checked in Examples 1 to 4 were subjected to deep sequencing by using HiSeq 4000 (Illumina).

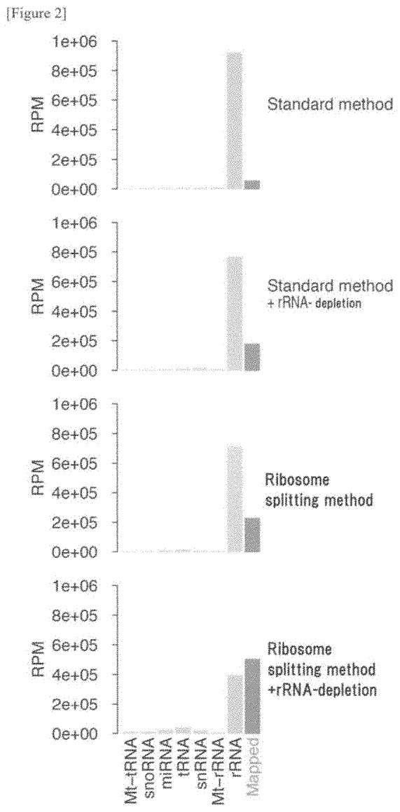

The results of the number of reads for the sequencing libraries prepared by the standard method, ribosome splitting method, standard method—rRNA depletion, and ribosome splitting method+rRNA depletion are shown in FIG. 2. “Mapped” in FIG. 2 refers to the number of reads mapped on the protein coding region (CDS), which corresponds to the number of ribosomes in mRNA.

In ribosome profiling, of the library prepared from HEK293 cells by the standard method, the reads from rRNA accounted for 92% [9.2×10⁵ reads per million (RPM)], and usable fraction of reads that were not originated from non-coding RNAs (such as rRNA, tRNA, Mt-rRNA, Mt-tRNA, snRNA, snoRNA, and miRNA) accounted for only 5.4% (0.54×10⁵ RPM) (FIG. 2. Standard method).

rRNA-subtraction oligonucleotides, which hybridize with rRNA and can be trapped on magnetic beads, have been used for depleting rRNA reads (Ingolia et al., 2009, Science, 324, 218-23; Weinberg et al, 2016, Cell Rep., 14, 1787-1799; McGlincy and Ingolia, 2017, Methods, 126, 112-129). This rRNA depletion using the rRNA-subtraction oligonucleotide reduced the rRNA contamination so that the read number of rRNA was 7.7×10⁵ RPM. 77% in the library, and increased the yield of reads from mRNA to 1.8×10⁵ RPM. 18% in the library (FIG. 2, Standard method—rRNA depletion).

On the other hand, the ribosome splitting method increased the yields of mRNA reads to 2.3×10⁵ RPM, 23% in the library (FIG. 2, Ribosome splitting method). Furthermore, the combination of the Ribosome splitting method with rRNA depletion gave further improvements of yield of reads from mRNA to 5.0×10⁵ RPM. 50% in the library (FIG. 2, Ribosome splitting method—rRNA depletion).

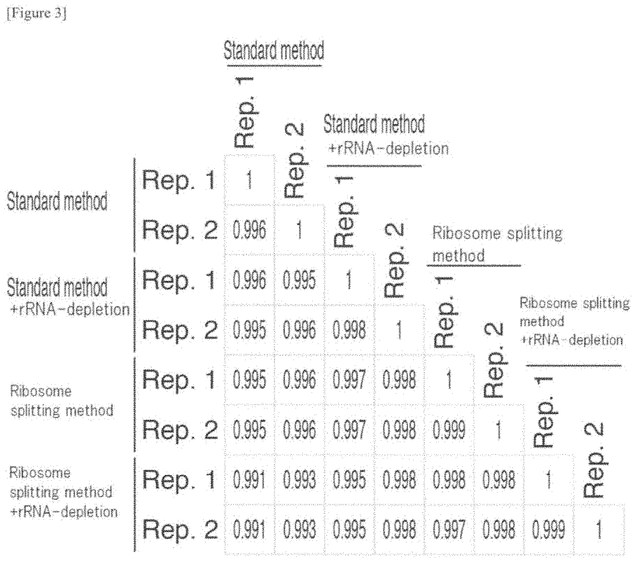

Ribosome profiling was performed for the libraries prepared by replicating twice by each of the standard method, the ribosome splitting method, the standard method—rRNA depletion, and the ribosome splitting method—rRNA depletion, and Pearson's correlation coefficient was calculated for the yields of mRNA reads obtained by each method and two repetitions of each method. Pearson's correlation coefficient is a dimensionless measure of the covariance, which is scaled such that it ranges from −1 to +1. A strong relationship is shown when the value is between 0.7 and 1. FIG. 3 shows the correlation coefficients. The yields of mRNA reads showed high reproducibility between them obtained by two repetitions of each method, and the high correlation of the data was observed even for different strategies of the above four methods.

### Materials

HEK293 cells (ADCC. cat. no. CRL-1573)

DMEM (1×) GlutaMAX-1 (Thermo Fisher Scientific. cat. no. 10566-016), with 10% FBS before use.

0.05% Trypsin-EDTA (Thermo Fisher Scientific, cat. no. 25300-54)

Cycloheximide 100 mg/ml (Sigma/Aldrich, cat. no. (74859-1 ML)

D-PBS (−)(1×) (Nacalai Tesque, cat. no. 14249-24)

RNase-free water, molecular biology grade (Millipore, cat. no. H20MB1001) or (Thermo Fisher Scientific, cat. no. 10977-015)

1 M Tris-HCl pH 7.5, molecular biology grade (Wako Pure Chemical Industries, Ltd., cat. no. 318-90225)

5 M NaCl, molecular biology grade (Nacalai Tesque, cat. no. 06900-14)

1 M MgCl₂, molecular biology grade (Nacalai Tesque, cat. no. 20942-34)

Turbo DNase, 2 U/μl (Thermo Fisher Scientific, cat. no. AM2238)

Triton X-100, molecular biology grade (Nacalai Tesque, cat. no. 12967-32)

Qubit RNA BR Assay kit (Thermo Fisher Scientific, cat. no. Q10210)

SUPERase In, 20 U/μl (Thermo Fisher Scientific, cat. no. AM2694)

Sucrose, molecular biology grade (Wako Pure Chemical Industries, Ltd., cat. no. 198-13525)

3 M NaOAc pH 5.2, molecular biology grade (Nacalai Tesque, cat. no. 06893-24)

RNase I. 10 U/μl (Epicentre, cat. no. N690IK)

13×56 mm Polycarbonate ultracentrifuge tube (Beckman Coulter, cat. no. 362305)

Direct-zol RNA MicroPrep (Zymo Research, cat. no. R2062)

TRIzol (Thermo Fisher Scientific, cat. no. 15596018) or other Direct-zol compatible reagent

Ethanol, molecular biology grade (Wako Pure Chemical Industries, Ltd., cat. no. 054-07225)

Isopropanol, molecular biology grade (Wako Pure Chemical Industries, Ltd., cat. no. 168-21675)

GlycoBlue, 15 mg/ml (Thermo Fisher Scientific, cat. no. AM9515)

0.5 M EDTA, molecular biology grade (Wako Pure Chemical Industries. Ltd., cat. no. 311-90075)

2 RNA Loading Buffer without Ethidium Bromide (Wako Pure Chemical Industries, Ltd., cat. no. 182-02571)

SuperSepRNA, 15%, 17 well (Wako Pure Chemical Industries, Ltd., cat. no. 194-15881)

10,000× SYBR Gold (Thermo Fisher Scientific, cat. no. S11494)

UltraPure 10% SDS (Thermo Fisher Scientific, cat. no. 15553-027)

T4 Polynucleotide kinase (New England Biolabs, cat. no. M0201S). Supplied with 10× T4 polynucleotide kinase buffer.

T4 RNA Ligase 2, truncated K227Q (New England Biolabs, cat. no. M0351S). Supplied with PEG 8000 50% w/v and 10× T4 RNA ligase buffer.

Preadenylated linkers at 20 μM

Oligo Clean & Concentrator (Zymo Research, cat. no. D4060)

10 mM dNTP mix (New England Biolabs, cat. no. N0447L)

ProtoScript II (New England Biolabs, cat. no. M0368L). Supplied with 5× first-strand buffer and 0.1 M DTT.

1 M Sodium hydroxide (Nacalai Tesque. cat. no. 37421-05)

CircLigaseII ssDNA ligase (Epicentre, cat. no. CL9025K). Supplied with 10× CircLigaseII buffer, 5 M Betaine, and 50 mM MnCl₂.

Phusion polymerase (New England Biolabs, cat. no. M0530S). Supplied with 5× HF buffer.

Gel Loading Dye. Purple (6×) (New England Biolabs, cat. no. B7024S)

SuperSep DNA, 15%, 17 well (Wako Pure Chemical Industries, Ltd., 190-15481)

DNA-1000 kit (SHIMAZU BIOTECH)

GelStar Nucleic Acid Gel Stain 10,000 ·(LONZA, cat. no. 50535)

100 bp DNA Ladder (TAKARA BIO, cat. no. 3407A)

NucleoSpin Gel and PCR Clean-up (TAKARA, cat. no 740609.250)

### Equipments

DNA LoBind Tube 1.5 mL (Eppendorf, cat. no. 022431021)

8-Strip PCR tube with lid (BIO-BIK, cat. no. 3247-00)

Nunc 50 mL Conical Sterile Polypropylene Centrifuge Tubes (Thermo Fisher Scientific, cat. no. 339652)

Eppendorf Tubes 5.0 mL (Eppendorf, cat. no. 0030122313)

Low retention filter tips (Greiner Bio-One, cat. nos. 771265, 773265, 738265, and 750265)

Short 10 μl filter tips (Watson, cat. no. 1252-207CS)

Wide Bore 200 μl filter tips (Axygen, cat. no. TF-205-WB-R-S)

Gel loading 20 μl filter tips (Thermo Fisher Scientific, cat. no. 2155P)

Refrigerated microcentrifuge (TOMY, cat. no. MX-307)

Qubit 2.0 Fluorometer (Thermo Fisher Scientific)

Optima MAX-TL Ultracentrifuge (Beckman, cat. no. A95761)

TLA 110 rotor (Beckman, cat. no. 366735)

Dry block heater (Major science, cat. no. MC-0203)

EasySeparator (Wako Pure Chemical Industries, Ltd. cat. no. 058-07681)

Electrophoresis power supply (Amercham Biosciences, cat. no. EPS 301)

Blue light illuminator and orage filter cover (NA). A standard UV transilluminator can be used instead.

Razors (Feather, cat. no. FAS-10) or (Feather, cat. no. No 11 stainless steel)

Spin-X centrifuge tube filter 0.22 μM (Costar, cat. no. 8160)

Thermal cycler (Applied Biosystems, cat. no. 2720)

DynaMag-2 separation rack (Thermo Fisher Scientific, cat. no. 12321D)

MixMate (Eppendorf)

MultiNA (SHIMADZU BIOTECH)

Disposable homogenizer pestle R-1.5 (ASONE, cat. no. 1-2955-01)

1. A method for producing a non-ribosomal RNA-containing sample, which comprises splitting subunits of ribosomes and mRNAs in a sample containing mRNAs and ribosomes, and removing the split subunits of ribosomes.

2. The method for producing a non-ribosomal RNA-containing sample according to claim 1, which further comprises degrading RNAs or fragmenting RNAs in a sample containing mRNAs and ribosomes.

3. The method for producing a non-ribosomal RNA-containing sample according to claim 1, wherein the splitting subunits of ribosomes and mRNAs is performed by using a chelating agent.

4. The method for producing a non-ribosomal RNA-containing sample according to claim 1, wherein the removing the split subunits of ribosomes is performed by ultrafiltration.

5. A method for analyzing a non-ribosomal RNA, which comprises obtaining a non-ribosomal RNA-containing sample by performing the method for producing a non-ribosomal RNA-containing sample according to claim 1, and
sequencing RNAs in the non-ribosomal RNA-containing sample.

6. A kit for use in performing the method for producing a non-ribosomal RNA-containing sample according to claim 1, which comprises a reagent for splitting subunits of ribosomes and mRNAs, and a remover for removing subunits of ribosomes.
